(19) United States
(12) Patent Application Publication
(10) Pub. No.: US 2020/0155532 A1
(43) Pub. Date: May 21, 2020

(54) METHOD FOR TREATING VIRAL INFECTIONS WITH LOPERAMIDE

(71) Applicant: Bruce Kent Evans, Eustis, FL (US)

(72) Inventor: Bruce Kent Evans, Eustis, FL (US)

(21) Appl. No.: 16/196,467

(22) Filed: Nov. 20, 2018

(51) Int. Cl. A61K 31/451

(52) U.S. Cl. CPC A61K 31/451

(57) ABSTRACT

A method of treating viral infections, the method includes administering to a patient an effective amount of a drug having loperamide as an active ingredient; specifically, wherein the viral infection is herpes-related.

### BACKGROUND OF THE INVENTION

The present invention relates to methods of treating viral infections and, more particularly, to a new use of loperamide as an anti-viral agent in treating herpes flare-ups and outbreaks.

The most recent herpes group of viral infections has been a curse: they are very painful and are transmitted easily. All current herpes antivirals (such as Zovirox™ and Zostervax™) are medically available only through prescription. Another reason they are expensive is that, like most pharmaceuticals, they are held for years under exclusive patent. Furthermore, current herpes antivirals are only moderately effective and do not mitigate pain. In effect, there is current medication that treats both the duration and pain of the herpes group of viral infections. and the current regimen of herpes antivirals is long, painful, and costly.

As can be seen, there is a need for a new use of loperamide as an anti-viral agent for treating both the duration and pain of the herpes group of viral infections.

Loperamide, sold under the brand name Imodium™ by Johnson and Johnson, among others, is a medication used to decrease the frequency of diarrhea. It is often used for this purpose in gastroenteritis, inflammatory bowel disease, and short bowel syndrome. The medication is taken by mouth.

Loperamide in an over the counter (OTC), non-patent protected, low-cost medicament available to everyone that the inventor has discovered a secondary medical application for treating viral infections quickly and effectively; specifically, herpes viral infections. The new use of loperamide for treating herpes outbreaks has shown to stop the pain within 30 minutes and stop the virus's activity: achieving a full recovery is three days.

Just as aspirin was used as an anti-inflammatory and then a blood thinner, Loperamide stops diarrhea and has a more formidable use for a much larger demographic.

Finally, loperamide has a very low incidence of drug interactions and may be trusted to continue that history when used as a treatment for herpes viral infections, such as Herpes Simplex.

### SUMMARY OF THE INVENTION

In one aspect of the present invention, a method of treating viral infections includes administering to a patient an effective amount of a drug comprising loperamide as an active ingredient.

In another aspect of the present invention, the new use is applied during a herpes-related outbreak or flare-up.

These and other features, aspects and advantages of the present invention will become better understood with reference to the following drawings, description and claims.

### BRIEF DESCRIPTION OF THE DRAWINGS

FIG. 1 is a flowchart of an exemplary embodiment of the present invention, demonstrating use thereof; and

FIG. 2 is a flowchart of an exemplary embodiment of the present invention, demonstrating production thereof.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a method of treating viral infections, the method includes administering to a patient an effective amount of a drug having loperamide as an active ingredient; specifically, wherein the viral infection is herpes-related.

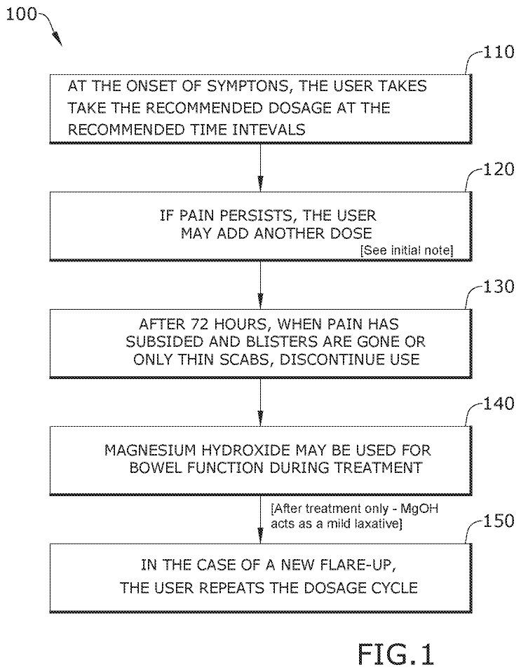

Referring to FIG. 1, the present invention may include a new use of loperamide 100 as an anti-viral agent. Herpes, which erupts during stress and may include repeated flare-ups over, sometimes, weeks may be treated with the new use of loperamide 100 disclosed herein, which tends to eliminate this recycling when taken at every flare-up. Specifically, the new use 100 of loperamide as an anti-viral agent may have the following steps: at the onset of symptoms, the user takes the recommended amount at the recommended time intervals, such as two 2 mg loperamide tablets four or five times a day, in step 110; if pain persists, the user may review the dosage, in step 120; after 72 hours, when pain has subsided and blisters are gone or only thin scabs, the patient may discontinue use in step 130; magnesium hydroxide may be used for bowel function during treatment, in step 140; and in the case of a new flare-up, the user-patient may repeat the dosage cycle in step 150.

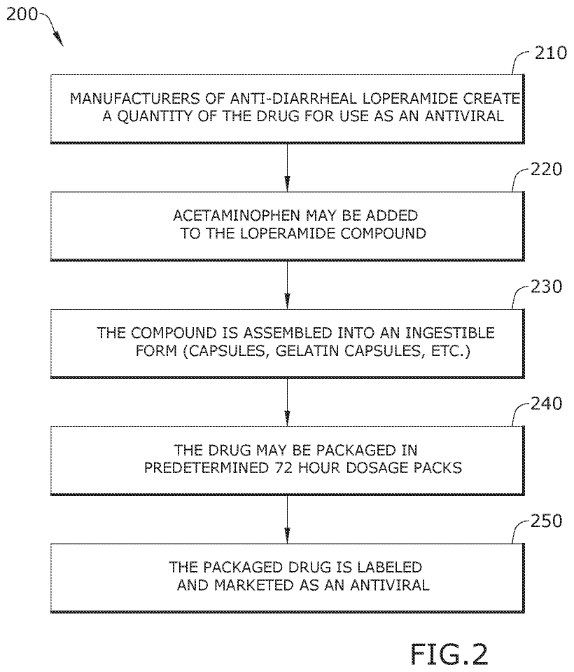

Referring to FIG. 2, the method of production of loperamide as an anti-viral agent 200 may have the following steps: of the manufactures of anti-diarrheal loperamide create a quantity of the drug for use as an anti-viral, in step 210; acetaminophen may be added to the loperamide compound, in step 220; the compound is assembled into an ingestible form (capsules, gelatin capsules, etc.), in step 230; the drub may be packaged in predetermined 72-hour dosage packs (to avoid the risk of over-ingestion), in step 240; and the packaged drug may be labeled and marketed as antiviral, in step 250.

The antiviral effects upon the herpes group of viral infections is not noted in pharmacology studies by Johnson and Johnson or any other company. The catalysis of antiviral agents is, therefore, unknown.

It should be understood, of course, that the foregoing relates to exemplary embodiments of the invention and that modifications may be made without departing from the spirit and scope of the invention as set forth in the following claims.

1. A method of treating a duration of a herpes infection, the method comprising administering to a patient an effective amount of a drug comprising loperamide as an active ingredient.

2. (canceled)

3. The method of claim 1, wherein the administering includes 4 mg of loperamide every four hours for up to three days.

4. The method of claim 3, further comprising discontinue administering the drug comprising loperamide as an active ingredient when one or more blisters of the herpes infection are gone or reduced to scabs.
